(19) United States
(12) Patent Application Publication
(10) Pub. No.: US 2002/0192671 A1
(43) Pub. Date: Dec. 19, 2002

(54) Method and system for predicting the biological activity, including toxicology and toxicity, of substances

(76) Inventors: Arthur L. Castle, Washington, DC (US); Michael Elashoff, Germantown, MD (US)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/052,547

(22) Filed: Jan. 23, 2002

Related U.S. Application Data
(60) Provisional application No. 60/263,161, filed on Jan. 23, 2001.

(51) Int. Cl. C12Q 1/68; G06F 19/00; G01N 33/48; G01N 33/50

(52) U.S. Cl. 435/6; 702/20

(57) ABSTRACT

A method for assessing toxicity and toxicology of a substance is disclosed comprising: exposing a set of at least two genes to the substance; monitoring the response of each gene in the set of genes to the substance; analyzing the variance of the response to the substance for each gene using contrast analysis; constructing a summary score for each gene in the set of genes; performing a logistic regression analysis upon the summary scores; and using the results of the logistic regression analysis to provide a predictive model regarding the toxicity and toxicology of the substance.

### CROSS-REFERENCE TO RELATED APPLICATIONS

This application claims priority to and incorporates herein by reference in its entirety U.S. Provisional Patent Application: No. 60/263,161 entitled “A Method And System For Predicting The Biological Activity, Including Toxicology And Toxicity, Of Substances,” filed Jan. 23, 2001.

### BACKGROUND OF THE INVENTION

1. Field of the Invention

The present invention relates generally to a system and method for predictively assessing the biological activity of a substance, and, more specifically, the toxicity and toxicology of a substance, utilizing a multi-variate statistical analysis of multiple gene expression patterns in response to that substance.

2. Description of the Related Art

At least 55,000 chemicals are presently produced in the United States and over 2,000 new chemicals are introduced into the market each year. Very few of these chemicals have been comprehensively tested for acute or chronic toxicity. For example, less than 1 percent of commercial chemicals have undergone complete health hazard assessment.

The Environmental Protection Agency (“EPA”) has the authority to require toxicological testing of a chemical prior to commercial production, but that authority is rarely invoked. Less than 10 percent of new chemicals are subjected to detailed review by the EPA. In the interest of cost and speedy access to the market, the EPA often uses the toxicity of previously tested homologous compounds to gauge the toxicity of a new chemical.

The potential toxicity of new drugs is monitored by the Food and Drug Administration (“FDA”). For a New Drug Application (NDA), the FDA typically requires a large battery of toxicity, carcinogenicity, mutagenicity and reproduction/fertility tests in at least two species of live animals. These tests are required to last up to one year. The costs involved in completing these tests is enormous. For example, a typical 90-day exposure toxicity test in rats costs approximately $100,000. A two year toxicity test in rats costs approximately $800,000 (Casarett and Doull's Toxicology, 4th Edition, M. O. Amdur et al., eds. Pergamon Press, New York, N.Y., p. 37 (1991)).

In addition, toxicity testing is a necessary and time-consuming part of the pharmaceutical drug development pipeline. A research tool that would allow for accurate predictions regarding the toxicity of a substance, such as a lead drug candidate, without conducting costly and time-consuming in vivo studies would greatly facilitate pharmaceutical research.

Besides cost, animal testing also presents disadvantages in terms of time, animal suffering and accuracy. Typical toxicity tests are divided into three stages: acute, short term and long term. Acute tests, which determine the LD₅₀ of a compound (the dose at which 50% of test animals are killed), require some 60-100 animals and a battery of tests for determining LD₅₀, dose-response curves and for monitoring clinical end points, other than death. Short term tests usually involve at least 24 dogs and 90 rats and last from 90 days in rats to 6-24 months in dogs. Body weight, food consumption, blood, urine and tissue samples are frequently measured in the short-term tests. In addition, dead animals are subjected to post-mortem examinations. Long term tests are similar to short term tests, but last 2 years in rats and up to 7 years in dogs or monkeys.

Animal testing has come under criticism by animal rights activists and the general public because of the severe suffering inflicted on the animals. Moreover, recent evidence calls into question the accuracy of animal testing. For example, variables, such as animal diet, may impair the predictability of animal tests in determining carcinogenic properties. P. H. Abelson, “Diet and Cancer in Humans and Rodents”, Science, 255, p. 141 (1992). Prior determinations on dioxin toxicity, based on guinea pig testing, are now being reevaluated. B. J. Culliton, “U.S. Government Orders New Look At Dioxin”, Nature, 352, p. 753 (1991); L. Roberts, “More Pieces in the Dioxin Puzzle”, Research News, October, 1991, p. 377. It is therefore apparent that there is an urgent need for a quick, inexpensive and reliable alternative to toxicity testing in animals.

Several short-term alternative tests are available. For example, the Ames Assay detects carcinogens which cause genetic reversion of mutant strains of Salmonella typhimurium.

U.S. Pat. No. 5,736,35, issued to Fielden, et al., discloses a method of determining the toxicity of a fluid sample comprising mixing the sample with a suspension of light emitting organisms; monitoring the light output of the mixture continually over a period of time; and providing an assessment of toxicity based on changes in light transmission.

U.S. Pat. No. 5,702,915, issued to Miyamoto, discloses a biosensor for detecting the toxicity of a sample which includes a solid-state area image pickup element, a culture container positioned on an upper surface of a light-receiving portion of the element, a cell cultured in the culture container, and culture medium for growing the cell.

U.S. Pat. No. 5,589,337, issued to Farr, discloses diagnostic kits for determining the toxicity of a compound employing a plurality of bacterial hosts, each of which harbors a DNA sequence encoding a different stress promoter fused to a gene which encodes an assayable product.

U.S. Pat. No. 5,569,580, issued to Young, discloses a method for the in vitro testing of chemicals to determine toxicity using hyperactivated rabbit spermatozoa.

U.S. Pat. No. 6,160,105, issued to Cunningham, et al., discloses methods for screening compounds for toxicological responses employing a composition comprising a plurality of polynucleotide targets used as hybridizable array elements in a microarray.

However, these assays suffer from a significant shortcoming in that none of these tests permit a predictive assessment of the biological activity, toxicology, and toxicity of a substance

As examples of substances with toxic effects, carbon tetra chloride (CCl₄), which causes hepatitis, when introduced into liver cells of a mature rat, produces a leak-out and change of cell morphology of enzymes such as glutamic-pyruvic transaminase (GPT), glutamicoxaloacetic transaminase (GOT) and lactate dehydrogenase (LDH). Based on this fact, there has been proposed a possibility of detecting hepatotoxin.

Benzo(a)pyrene is a known rodent and likely human carcinogen and is the prototype of a class of compounds, the polycyclic aromatic hydrocarbons. It is metabolized by several forms of cytochrome P450 and associated enzymes to both activated and detoxified metabolites Degawa et al. (1994) Cancer Res. 54: 4915-4919). The ultimate metabolites are the bay-region diol epoxide, benzo(a)pyrene-7,8-diol-9,10-epoxide (BPDE) and the K-region diol epoxide, 9-hydroxy benzo(a)pyrene-4,5-oxide, which have been shown to cause DNA adduct formation (alkylation of guanine bases). DNA adducts have been shown to persist in rat liver up to 56 days following treatment with benzo(a)pyrene at a dose of 10 mg/kg body weight 3 times per week for 2 weeks (Qu and Stacey, (1996) Carcinogenesis 17: 53-59).

Acetaminophen is a widely-used analgesic. It is metabolized by specific cytochrome P450 isozymes with the majority of the drug undergoing detoxification by glucuronic acid, sulfate and glutathione conjugation pathways (Chen et al. (1998) Chem. Res. Toxical 11: 295-301). However, at high non-therapeutic doses, acetaminophen can cause hepatic and renal failure by being metabolized to an active intermediate, N-acetyl-p-benzoquinone imine (NAPQI). NAPQI then binds to sulfhydryl groups of proteins causing their inactivation and leading to subsequent cell death (Kroger et al. Gen. Pharmacol. (1997 28: 257-263).

Clofibrate is an antilipidemic drug which lowers elevated levels of serum triglycerides. In rodents, chronic treatment produces hepatomegaly, an increase in hepatic peroxisomes Lock et al. (1989) Ann. Rev. Pharmacol. Toxicol. 29: 145-163). Clofibrate has been shown to increase levels of cytochrome P450 4A and reduce the levels of P450 4F (Kawashima et al. (1997) Arch. Biochem. Biophys. 347: 148-154). It is also involved in transcription of -oxidation genes as well as induction of peroxisome proliferator activated receptors Kawashima supra.

Thus, there remains a need for an efficient and effective system and method for predictively assessing the biological activity of a substance, and, more specifically, the toxicity and toxicology of a substance, utilizing a multi-variate statistical analysis of multiple gene expression patterns in response to that substance.

### BRIEF SUMMARY OF THE INVENTION

It is a feature and advantage of the present invention to provide an improved system and method for predictively assessing the biological activity of a substance.

It is a further feature and advantage of the present invention to provide an improved system and method for predictively assessing the toxicology of a substance.

It is a further feature and advantage of the present invention to provide an improved system and method for predictively assessing the toxicity of a substance.

To achieve the stated and other features, advantages and objects, an embodiment of the present invention provides an improved system and method for predictively assessing the biological activity of a substance, and, more specifically, the toxicity and toxicology of a substance, utilizing a multi-variate statistical analysis of multiple gene expression patterns in response to that substance.

This system and method employs the use of gene expression microarrays. For example, microarrays consisting of full length genes or gene fragments on a substrate may be formed. These arrays can then be tested with samples treated with a substances to elucidate the gene expression pattern associated with treatment with the substance. This gene pattern can be compared with gene expression patterns of compounds associated with known toxicological responses.

The present invention provides also systems and methods for the screening, preferably in a microarray format, of compounds and therapeutic treatments for toxicological effects.

Additional objects, advantages and novel features of the invention will be set forth in part in the description which follows, and in part will become more apparent to those skilled in the art upon examination of the following, or may be learned by practice of the invention.

### BRIEF DESCRIPTION OF THE DRAWINGS

FIGS. 1a, 1b, 1c, and 1d present four preferred patterns for illustrating the response of a gene or set of genes to a chemical.

FIG. 2 presents the principal component analysis of the CCl₄ data.

FIG. 3 presents the principal component analysis of the APAP data.

FIG. 4 presents the APAP predictive similarity model.

FIG. 5 presents the CCl₄ predictive similarity model.

### DETAILED DESCRIPTION OF THE INVENTION

The present invention pertains to the development of a method for assessing the toxicity and toxicology of a substance. In one preferred embodiment of the present invention, for each study, one derives a predictive model relating gene expression to toxicity such that it can be used to screen compounds. One then compares and cross-validates various models with other toxicological studies so as to refine the models.

It will be appreciated that in such a study, one relies upon various study designs. These, preferably, include time (one or more time points); treatment (one or more doses); and vehicle (which may differ from study to study).

In a preferred embodiment of the present invention a minimum of three animals are tested per group.

It will be further appreciated that treatments related to one or more toxic pathways may be explored, which treatments may differ from study to study.

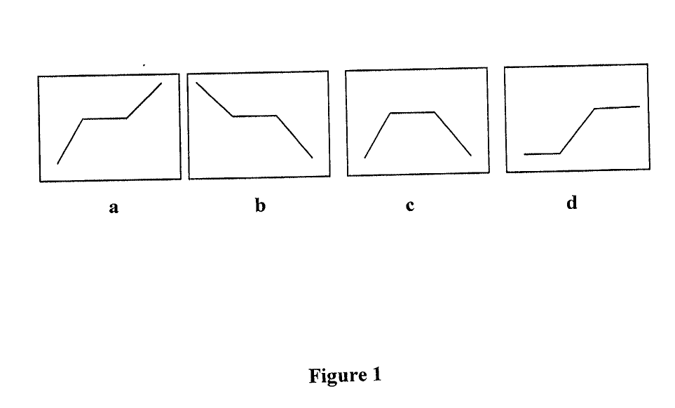

An aspect of the present invention is an analysis of the variance for each gene contrast analysis. In this gene contrast analysis, the response of a gene or set of genes is monitored upon exposure to a chemical. In one preferred embodiment, the response of a gene or set of genes to a chemical can be fitted into one of four patterns illustrated in FIGS. 1a, 1b, 1c, and 1d. In this preferred embodiment, upon classification into one of these four groups, an analysis is then performed which categorizes the gene contrast analysis as one of four summary scores. These summary scores are then subjected to logistic regression analysis, furnishing a predictive model.

In another preferred embodiment of the present invention, the input data for the analysis of the variance for each gene contrast analysis is the average difference for all samples and all genes. In yet another preferred embodiment of the present invention, the analysis fits two factors (for example, time and dose) in an analysis of variance (ANOVA) methodology, using contrast analysis to assign each gene to a pattern. In still another preferred embodiment, the output comprises a correlation of a list of patterns and a list of genes within each pattern, coupled with a measure of the fit.

In still another preferred embodiment of the present invention, responses of a gene or set of genes to a chemical that fit into patterns corresponding to either FIGS. 1a or 1b are subjected to analysis which categorizes the gene contrast analysis as one of four summary scores. In such an embodiment, the input data are genes selected from patterns that are biologically relevant to the toxicological process; the analysis is performed for all samples on selected genes; and the output data comprises summary scores for each sample.

In a further preferred aspect of this embodiment, the summary scores are subjected to logistic regression analysis, resulting in a predictive model. In this aspect of the embodiment, the input data are the summary scores per sample, which is an indicator for each sample; the analysis is a logistic regression analysis mapping the summary scores to a 0 to 1 scale of toxicity; and the out put data are one are more mathematical formulae that converts a column of average differences into a single 0 to 1 toxicological score for a sample.

It will be appreciated that another preferred aspect of the present invention is an assessment of false positive and false negative rates so as to test the validity of the predictive model.

Another aspect of the present invention is the correlation of a predictive model with results obtained from other studies. Thus, preferably, one seeks validation of each model with vehicles and toxins from other models. In this mode, non-similar toxins should score low; similar toxins should score high; and vehicles should score low regardless of vehicle type.

In correlating these other studies, one preferably compare gene lists for patterns of interest between studies of related compounds to arrive at a consensus set of genes involved in a toxicological response.

In another preferred embodiment of the present invention, the goal of the method for assessing the toxicity and toxicology of a substance is to use gene expression to predict whether a compound has a high probability of being toxic at a given dose. In this preferred embodiment, patterns of gene expression can be compared against know “toxic” patterns and a similarity score calculated. Preferably, the methodology associated with this preferred embodiment includes identification of gene expression patterns associated with toxicity; quantification of this association; development of a statistical inference of similarity; and validation of results.

It will be appreciated that in such a modeling, there cam be a number of different types of markers, including general markers, group markers (for example, cholestasis, necrosis, stenosis), and compound specific markers.

It will appreciated that there are preferred model attributes. These include: time stability (must be able to predict toxicity over an extended time range); dose dependency (should only score toxic doses of compounds); vehicle independence (should not be sensitive to type of vehicle used); predictable (based on statistical inference with known false positive rate); and powerful (false negative rates should be low enough that singeltons or low number of replicates can adequately predict toxicity).

In another preferred embodiment of the present invention, there are various stages of model development. These, preferably, include: selection (determination of relevant expression patterns that are time stable and dose dependent); quantification production of composite measures that define patterns); prediction (use of composite measures to assign probability of patterns being the same); and validation (ability to provide statistical measures of model accuracy).

It will be recognized that the present invention enables one to develop models for key compounds; cross-validate each model; identify false positives and false negatives; provide positive crossover; reduce models to best set of toxic markers; and predict the toxicity of unknown compounds.

The expression similarity profiling for predictive toxicology models are developed based on the gene expression patterns of known toxic substances. The gene expression patterns of unknown chemicals are compared against these known patterns and a probability of similar toxic profile is produced. Recognizing these gene expression patterns and producing a single predictive score from thousands of individual measurements involves the use of multiple established techniques in a non-obvious linear sequence.

These techniques provide for selection of time-stable and dose-dependent toxic gene expression profiles via contrast analysis and selection of thousands of variables into one or more composite variables via principal component analysis (PCA).

Use of composite variables allows one to make a predictive composite measure via logistic regression. In addition, the present invention provides for validation of the model by testing both known toxic and non-toxic substances using this composite measure.

The ability to tell whether a chemical compound has a high probability of being toxic based on its gene expression profile. This is a critical issue for the safety of potential pharmaceutical compounds

The gene expression pattern caused by an unknown substance will be entered into a series of formulas. These formulas will then predict the likelihood of toxicity on a 0 to 1 scale, 0 being the highest confidence in safety and 1 being the highest confidence in toxicity

In one aspect, the invention provides a method for screening a compound for a toxicological effect. The method comprises selecting a plurality of polynucleotide targets, wherein the polynucleotide targets have first gene expression levels altered in tissues treated with known toxicological agents when compared with untreated tissues. Some of the first gene expression levels may be upregulated and others downregulated when associated with a toxicological response. A sample is treated with the compound to induce second gene expression levels of a plurality of polynucleotide probes. Then first and second gene expression levels are compared to identify those compounds that induce expression levels of the polynucleotide probes that are similar to those of the polynucleotide targets and the similarity or expression levels correlates with a toxicological effect of the compound.

Preferred tissues are selected from the group consisting of liver, kidney, brain, spleen, pancreas and lung. Preferred toxicological agents are acetaminophen and other compounds with a similar mechanism of action.

Alternatively, the invention provides methods for screening a therapeutic treatment for a toxicological effect or for screening a sample for a toxicological response to a compound or therapeutic treatment.

In another aspect, the invention provides methods for preventing a toxicological response by administering complementary nucleotide sequences against one or more selected upregulated polynucleotide targets or a ribozyme that specifically cleaves such sequences. Alternatively, a toxicological response may be prevented by administering sense nucleotide sequences for one or more selected downregulated polynucleotide targets.

In yet another aspect, the invention provides methods for preventing a toxicological response by administering an agonist which initiates transcription of a gene comprising a downregulated polynucleotide of the invention. Alternatively, a toxicological response may be prevented by administering an antagonist which prevents transcription of a gene comprising an upregulated polynucleotide of the invention.

Oligonucleotide probes have long been used to detect complementary nucleic acid sequences in a nucleic acid of interest (the “target” nucleic acid) and have been used to detect expression of particular genes (e.g., a Northern Blot). In some assay formats, the oligonucleotide probe is tethered, i.e., by covalent attachment, to a solid support, and arrays of oligonucleotide probes immobilized on solid supports have been used to detect specific nucleic acid sequences in a target nucleic acid. See, e.g., PCT patent publication Nos. WO 89/10977 and 89/11548. Others have proposed the use of large numbers of oligonucleotide probes to provide the complete nucleic acid sequence of a target nucleic acid but failed to provide an enabling method for using arrays of immobilized probes for this purpose. See U.S. Pat. Nos. 5,202,231 and 5,002,867 and PCT patent publication No. WO 93/17126.

The use of “traditional” hybridization protocols for monitoring or quantifying gene expression is problematic. For example two or more gene products of approximately the same molecular weight will prove difficult or impossible to distinguish in a Northern blot because they are not readily separated by electrophoretic methods. Similarly, as hybridization efficiency and cross-reactivity varies with the particular subsequence (region) of a gene being probed it is difficult to obtain an accurate and reliable measure of gene expression with one, or even a few, probes to the target gene.

The development of VLSIPS technology provided methods for synthesizing arrays of many different oligonucleotide probes that occupy a very small surface area. See U.S. Pat. No. 5,143,854 and PCT No. WO 90/15070. U.S. patent application Ser. No. 082,937, filed Jun. 25, 1993, describes methods for making arrays of oligonucleotide probes that can be used to provide the complete sequence of a target nucleic acid and to detect the presence of a nucleic acid containing a specific nucleotide sequence.

Prior to the present invention, however, it was unknown that high density oligonucleotide arrays could be used to reliably monitor message levels of a multiplicity of preselected genes in the presence of a large abundance of other (non-target) nucleic acids (e.g., in a cDNA library, DNA reverse transcribed from an mRNA, mRNA used directly or amplified, or polymerized from a DNA template). In addition, the prior art provided no rapid and effective method for identifying a set of oligonucteotide probes that maximize specific hybridization efficacy while minimizing cross-reactivity nor of using hybridization patterns (in particular hybridization patterns of a multiplicity of oligonucleotide probes in which multiple oligonucleotide probes are directed to each target nucleic acid) for quantification of target nucleic acid concentrations.

The present invention is premised, in part, on the discovery that microfabricated arrays of large numbers of different oligonucleotide probes (DNA chips) may effectively be used to not only detect the presence or absence of target nucleic acid sequences, but to quantify the relative abundance of the target sequences in a complex nucleic acid pool. In particular, prior to this invention it was unknown that hybridization to high density probe arrays would permit small variations in expression levels of a particular gene to be identified and quantified in a complex population of nucleic acids that out number the target nucleic acids by 1,000 fold to 1,000,000 fold or more.

Thus, this invention employs a method of simultaneously monitoring the expression (e.g. detecting and or quantifying the expression) of a multiplicity of genes. The levels of transcription for virtually any number of genes may be determined simultaneously. Typically, at least about 10 genes, preferably at least about 100, more preferably at least about 1000 and most preferably at least about 10,000 different genes are assayed at one time.

The method involves providing a pool of target nucleic acids comprising mRNA transcripts of one or more of said genes, or nucleic acids derived from the mRNA transcripts; hybridizing the pool of nucleic acids to an array of oligonucleotide probes immobilized on a surface, where the array comprises more than 100 different oligonucleotides, each different oligonucleqtide is localized in a predetermined region of said surface, the density of the different oligonucleotides is greater than about 60 different oligonucleotides per 1 cm², and the oligonucleotide probes are complementary to the mRNA transcripts or nucleic acids derived from the mRNA transcripts; and quantifying the hybridized nucleic acids in the array. In a preferred embodiment, the pool of target nucleic acids is one in which the concentration of the target nucleic acids (mRNA transcripts or nucleic acids derived from the mRNA transcripts) is proportional to the expression levels of genes encoding those target nucleic acids.

In a preferred embodiment, the array of oligonucleotide probes is a high density array comprising greater than about 100, preferably greater than about 1,000 more preferably greater than about 16,000 and most preferably greater than about 65,000 or 250,000 or even 1,000,000 different oligonucleotide probes. Such high density arrays comprise a probe density of generally greater than about 60, more generally greater than about 100, most generally greater than about 600, often greater than about 1000, more often greater than about 5,000, most often greater than about 10,000, preferably greater than about 40,000 more preferably greater than about 100,000, and most preferably greater than about 400,000 different oligonucleotide probes per cm². The oligonucleotide probes range from about 5 to about 50 nucleotides, more preferably from about 10 to about 40 nucleotides and most preferably from about 15 to about 40 nucleotides in length. The array may comprise more than 10, preferably more than 50, more preferably more than 100, and most preferably more than 1000 oligonucleotide probes specific for each target gene. Although a planar array surface is preferred, the array may be fabricated on a surface of virtually any shape or even a multiplicity of surfaces.

The array may further comprise mismatch control probes. Where such mismatch controls are present, the quantifying step may comprise calculating the difference in hybridization signal intensity between each of the oligonucleotide probes and its corresponding mismatch control probe. The quantifying may further comprise calculating the average difference in hybridization signal intensity between each of the oligonucleotide probes and its corresponding mismatch control probe for each gene.

The probes present in the high density array can be oligonucleotide probes selected according to the optimization methods described below. Alternatively, non-optimal probes may be included in the array, but the probes used for quantification (analysis) can be selected according to the optimization methods described below.

Oligonucleotide arrays for the practice of this invention are preferably synthesized by light-directed very large scaled immobilized polymer synthesis (VLSIPS) as described herein. The array includes test probes which are oligonucleotide probes each of which has a sequence that is complementary to a subsequence of one of the genes (or the mRNA or the corresponding antisense cRNA) whose expression is to be detected. In addition, the array can contain normalization controls, mismatch controls and expression level controls as described herein.

The pool of nucleic acids may be labeled before, during, or after hybridization, although in a preferred embodiment, the nucleic acids are labeled before hybridization. Fluorescence labels are particularly preferred and, where used, quantification of the hybridized nucleic acids is by quantification of fluorescence from the hybridized fluorescently labeled nucleic acid. Such quantification is facilitated by the use of a fluorescence microscope which can be equipped with an automated stage to permit automatic scanning of the array, and which can be equipped with a data acquisition system for the automated measurement recording and subsequent processing of the fluorescence intensity information.

In a preferred embodiment, hybridization is at low stringency (e.g., about 20° C. to about 50° C., more preferably about 30° C. to about 40° C., and most preferably about 37° C. and 6×SSPE-T or lower) with at least one wash at higher stringency. Hybridization may include subsequent washes at progressively increasing stringency until a desired level of hybridization specificity is reached.

The pool of target nucleic acids can be the total polyA.sup.+mRNA isolated from a biological sample, or cDNA made by reverse transcription of the RNA or second strand cDNA or RNA transcribed from the double stranded cDNA intermediate. Alternatively, the pool of target nucleic acids can be treated to reduce the complexity of the sample and thereby reduce the background signal obtained in hybridization. In one approach, a pool of mRNAs, derived from a biological sample, is hybridized with a pool of oligonucleotides comprising the oligonucleotide probes present in the high density array. The pool of hybridized nucleic acids is then treated with RNase A which digests the single stranded regions. The remaining double stranded hybridization complexes are then denatured and the oligonucleotide probes are removed, leaving a pool of mRNAs enhanced for those mRNAs complementary to the oligonucleotide probes in the high density array.

In another approach to background reduction, a pool of mRNAs derived from a biological sample is hybridized with paired target specific oligonucleotides where the paired target specific oligonucleotides are complementary to regions flanking subsequences of the mRNAs complementary to the oligonucleotide probes in the high density array. The pool of hybridized nucleic acids is treated with RNase H which digests the hybridized (double stranded) nucleic acid sequences. The remaining single stranded nucleic acid sequences which have a length about equivalent to the region flanked by the paired target specific oligonucleotides are then isolated (e.g. by electrophoresis) and used as the pool of nucleic acids for monitoring gene expression.

Finally, a third approach to background reduction involves eliminating or reducing the representation in the pool of particular preselected target mRNA messages (e.g., messages that are characteristically overexpressed in the sample). This method involves hybridizing an oligonucleotide probe that is complementary to the preselected target mRNA message to the pool of polyA.sup.+mRNAs derived from a biological sample. The oligonucleotide probe hybridizes with the particular preselected polyA.sup.+mRNA (message) to which it is complementary. The pool of hybridized nucleic acids is treated with RNase H which digests the double stranded (hybridized) region thereby separating the message from its polyA.sup.+tail. Isolating or amplifying (e.g., using an oligo dT column) the polyA.sup.+mRNA in the pool then provides a pool having a reduced or no representation of the preselected target mRNA message.

It will be appreciated that the methods of this invention can be used to monitor (detect and/or quantify) the expression of any desired gene of known sequence or subsequence. Moreover, these methods permit monitoring expression of a large number of genes simultaneously and effect significant advantages in reduced labor, cost and time. The simultaneous monitoring of the expression levels of a multiplicity of genes permits effective comparison of relative expression levels and identification of biological conditions characterized by alterations of relative expression levels of various genes. Genes of particular interest for expression monitoring include genes involved in the pathways associated with various pathological conditions (e.g., cancer) and whose expression is thus indicative of the pathological condition. Such genes include, but are not limited to the HER2 (c-erbB-2/neu) proto-oncogene in the case of breast cancer, receptor tyrosine kinases (RTKs) associated with the etiology of a number of tumors including carcinomas of the breast, liver, bladder, pancreas, as well as glioblastomas, sarcomas and squamous carcinomas, and tumor suppressor genes such as the P53 gene and other “marker” genes such as RAS, MSH2, MLH1 and BRCA1. Other genes of particular interest for expression monitoring are genes involved in the immune response (e.g., interleukin genes), as well as genes involved in cell adhesion (e.g., the integrins or selectins) and signal transduction (e.g., tyrosine kinases), etc.

In another embodiment, this invention provides for a method of selecting a set of oligonucleotide probes, that specifically bind to a target nucleic acid (e.g., a gene or genes whose expression is to be monitored or nucleic acids derived from the gene or its transcribed mRNA). The method involves providing a high density array of oligonucleotide probes where the array comprises a multiplicity of probes wherein each probe is complementary to a subsequence of the target nucleic acid. The target nucleic acid is then hybridized to the array of oligonucleotide probes to identify and select those probes where the difference in hybridization signal intensity between each probe and its mismatch control is detectable (preferably greater than about 10% of the background signal intensity, more preferably greater than about 20% of the background signal intensity and most preferably greater than about 50% of the background signal intensity). The method can further comprise hybridizing the array to a second pool of nucleic acids comprising nucleic acids other than the target nucleic acids; and identifying and selecting probes having the lowest hybridization signal and where both the probe and its mismatch control have a hybridization intensity equal to or less than about 5 times the background signal intensity, preferably equal to or less than about 2 times the background signal intensity, more preferably equal to or less than about 1 times the background signal intensity, and most preferably equal or less than about half the background signal intensity.

In a preferred embodiment, the multiplicity of probes can include every different probe of length n that is complementary to a subsequence of the target nucleic acid. The probes can range from about 10 to about 50 nucleotides in length. The array is preferably a high density array as described above. Similarly, the hybridization methods, conditions, times, fluid volumes, detection methods are as described above and herein below.

In addition, this invention provides for a composition comprising an array of oligonucleotide probes immobilized on a substrate, where the array comprises more than 100 different oligonucleotides and each different oligonucleotide is localized in a predetermined region of the solid support and the density of the array is greater than about 60 different oligonucleotides per 1 cm² of substrate. The oligonucleotide probes are specifically hybridized to one or more fluorescently labeled nucleic acids such that the fluorescence in each region of the array is indicative of the level of expression of each of a multiplicity of preselected genes. The array is preferably a high density array as described above and may further comprise expression level controls, mismatch controls and normalization controls as described herein.

Finally, this invention provides for kits for simultaneously monitoring expression levels of a multiplicity of genes. The kits include an array of immobilized oligonucleotide probes complementary to subsequences of the multiplicity of target genes, as described above. In one embodiment, the array comprises at least 100 different oligonucleotide probes and the density of the array is greater than about 60 different oligonucleotides per 1 cm² of surface. The kit may also include instructions describing the use of the array for detection and/or quantification of expression levels of the multiplicity of genes. The kit may additionally include one or more of the following: buffers, hybridization mix, wash and read solutions, labels, labeling reagents (enzymes etc.), “control” nucleic acids, software for probe selection, array reading or data analysis and any of the other materials or reagents described herein for the practice of the claimed methods.

With regard to the present invention, the phrase “massively parallel screening” refers to the simultaneous screening of at least about 100, preferably about 1000, more preferably about 10,000 and most preferably about 1,000,000 different nucleic acid hybridizations.

The terms “nucleic acid” or “nucleic acid molecule” refer to a deoxyribonucleotide or ribonucleotide polymer in either single-or double-stranded form, and unless otherwise limited, would encompass known analogs of natural nucleotides that can function in a similar manner as naturally occurring nucleotides.

An oligonucleotide is a single-stranded nucleic acid ranging in length from 2 to about 500 bases.

As used herein a “probe” is defined as an oligonucleotide capable of binding to a target nucleic acid of complementary sequence through one or more types of chemical bonds, usually through complementary base pairing, usually through hydrogen bond formation. As used herein, an oligonucleotide probe may include natural (i.e. A, G, C, or T) or modified bases (7-deazaguanosine, inosine, etc.). In addition, the bases in oligonucleotide probe may be joined by a linkage other than a phosphodiester bond, so long as it does not interfere with hybridization. Thus, oligonucleotide probes may be peptide nucleic acids in which the constituent bases are joined by peptide bonds rather than phosphodiester linkages.

The term “target nucleic acid” refers to a nucleic acid (often derived from a biological sample), to which the oligonucleotide probe is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding probe directed to the target. The term target nucleic acid may refer to the specific subsequence of a larger nucleic acid to which the probe is directed or to the overall sequence (e.g., gene or mRNA) whose expression level it is desired to detect. The difference in usage will be apparent from context.

“Subsequence” refers to a sequence of nucleic acids that comprise a part of a longer sequence of nucleic acids.

The term “complexity” is used here according to standard meaning of this term as established by Britten et al. Methods of Enzymol. 29:363 (1974). See, also Cantor and Schimmel Biophysical Chemistry: Part III at 1228-1230 for further explanation of nucleic acid complexity.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The phrase “hybridizing specifically to”, refers to the binding, duplexing, or hybridizing of a molecule only to a particular nucleotide sequence under stringent conditions when that sequence is present in a complex mixture (e.g., total cellular) DNA or RNA. The term “stringent conditions” refers to conditions under which a probe will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions will be those in which the salt concentration is at least about 0.01 to 1.0 M Na ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide.

The term “mismatch control” refers to a probe that has a sequence deliberately selected not to be perfectly complementary to a particular target sequence. The mismatch control typically has a corresponding test probe that is perfectly complementary to the same particular target sequence. The mismatch may comprise one or more bases. While the mismatch(s) may be locates anywhere in the mismatch probe, terminal mismatches are less desirable as a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions.

The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals may also be produced by intrinsic fluorescence of the array components themselves. A single background signal can be calculated for the entire array, or a different background signal may be calculated for each target nucleic acid. In a preferred embodiment, background is calculated as the average hybridization signal intensity for the lowest 5% to 10% of the probes in the array, or, where a different background signal is calculated for each target gene, for the lowest 5% to 10% of the probes for each gene. Of course, one of skill in the art will appreciate that where the probes to a particular gene hybridize well and thus appear to be specifically binding to a target sequence, they should not be used in a background signal calculation. Alternatively, background may be calculated as the average hybridization signal intensity produced by hybridization to probes that are not complementary to any sequence found in the sample (e.g. probes directed to nucleic acids of the opposite sense or to genes not found in the sample such as bacterial genes where the sample is mammalian nucleic acids). Background can also be calculated as the average signal intensity produced by regions of the array that lack any probes at all.

The term “quantifying” when used in the context of quantifying transcription levels of a gene can refer to absolute or to relative quantification. Absolute quantification may be accomplished by inclusion of known concentration(s) of one or more target nucleic acids (e.g. control nucleic acids such as Bio B or with known amounts the target nucleic acids themselves) and referencing the hybridization intensity of unknowns with the known target nucleic acids (e.g. through generation of a standard curve). Alternatively, relative quantification can be accomplished by comparison of hybridization signals between two or more genes, or between two or more treatments to quantify the changes in hybridization intensity and, by implication, transcription level.

An object of the present invention is to use gene expression to predict whether a compound has a high probability of being toxic at a given dose. In the system and method of the present invention, patterns of gene expression are compared against known “toxic” patterns and a similarity score calculated.

To accomplish those ends, the present invention provides a system and method for identifying gene expression patterns associated with various modes of toxicity; quantifying this association; develop a statistical inference of similarity; and validating the results of the toxicity test.

It will be appreciated that there are preferred characteristics of the present invention. These characteristics include time stability, dose dependence, vehicle independence, predictability, and power of the analysis. Specifically, the analysis should be time-stable in that it must be able to predict toxicity over an extended time range. In addition, the analysis should be dose-dependent such that it will only score toxic doses of compounds. Further, the analysis is preferably vehicle-independent, where it is not sensitive to the type of vehicle used. The analysis is also predictable, where the resultant statistical inference has a known false positive rate. Additionally, the analysis is powerful so that false negative rates are low enough that singletons or low number of replicates can adequately predict toxicity.

Two models, acetaminophen (APAP) and CCl₄ have been tested: With APAP, the tissues were assayed at 3, 6, and 24 hours, at three dosages (V, L, H dose). With CCl₄, the tissues were assayed at 1, 3, 6, 24, and 72 hours, at two dosages (V, H dose).In addition, various vehicle control samples were tested, including 74 samples of multiple types of vehicles, including oil, gum, and saline, at time points of 0, 1, 3, 6, 24, 48, 72 hours, and 7 days. In addition, other toxins were assayed, including methotrexate, thioacetamide, and CHCl₃.

For CCl₄ 147 patterns were observed, from which were selected 38 patterns with 816 genes, resulting in a prediction based on 4 principal components, with CCl₄ considered toxic at all time points.

For APAP 505 patterns were observed, from which were selected 28 patterns with 1024 genes, resulting in a prediction based on 8 principal components, with high doses of APAP considered toxic at all time points.

For CCl₄, there were 3 out of 74 (4.1%) false positives for all samples and 2 out of 53 (3.8%) for samples not in the model.

APAP, there were 3 out of 74 (4.1%) false positives for all samples and 3 out of 44 (6.8%) for samples not in the model.

In addition, there were detected 703 genes specific to CCl₄, 911 genes specific to APAP and 113 genes in common.

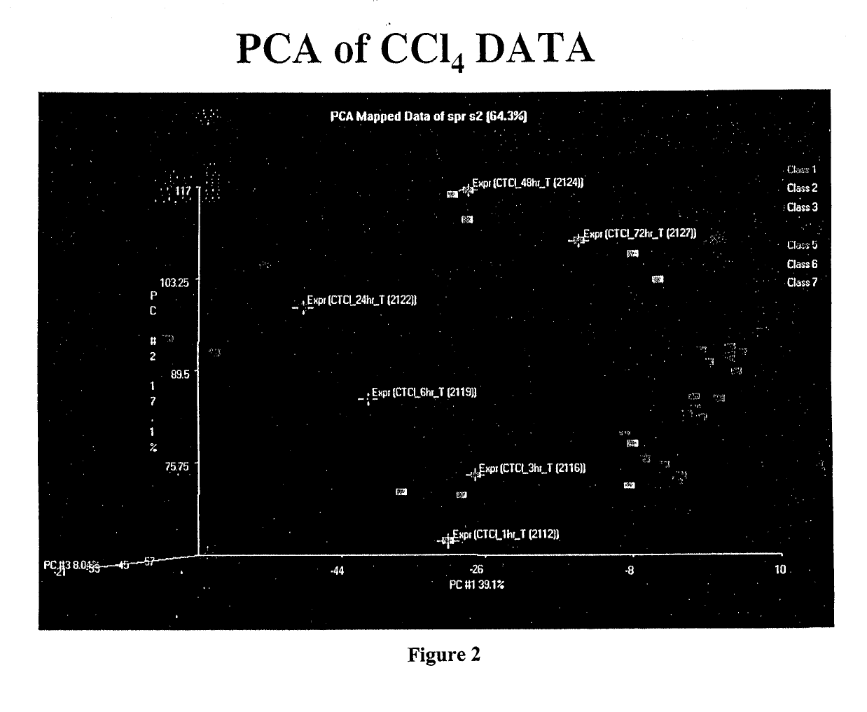

FIG. 2 presents the principal component analysis of the CCl₄ data.

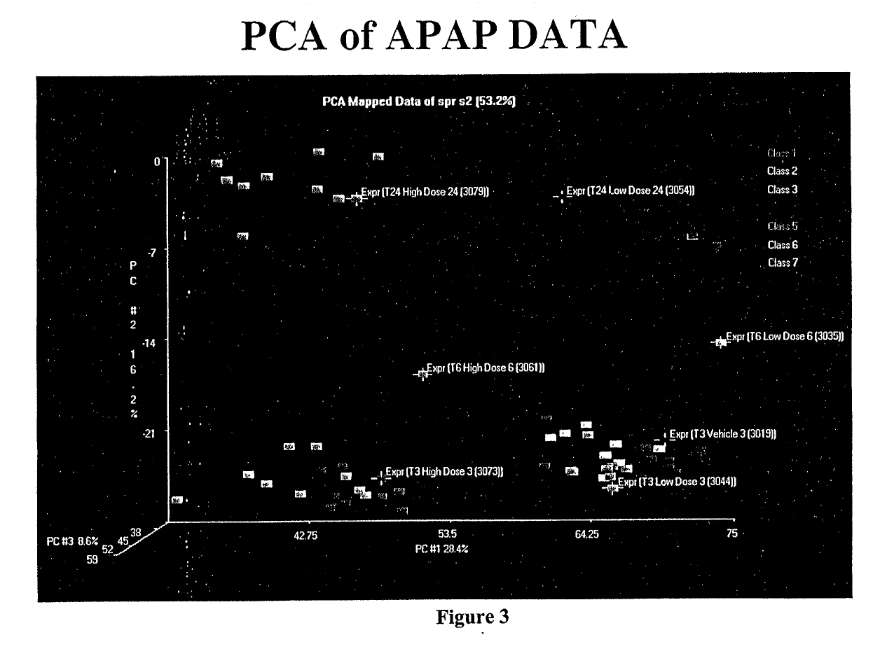

FIG. 3 presents the principal component analysis of the APAP data.

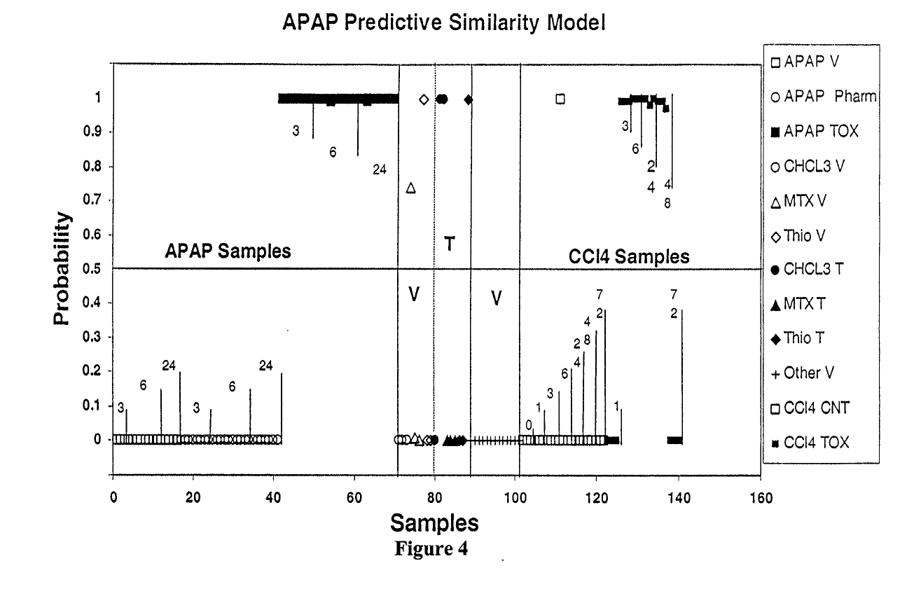

FIG. 4 presents the APAP predictive similarity model.

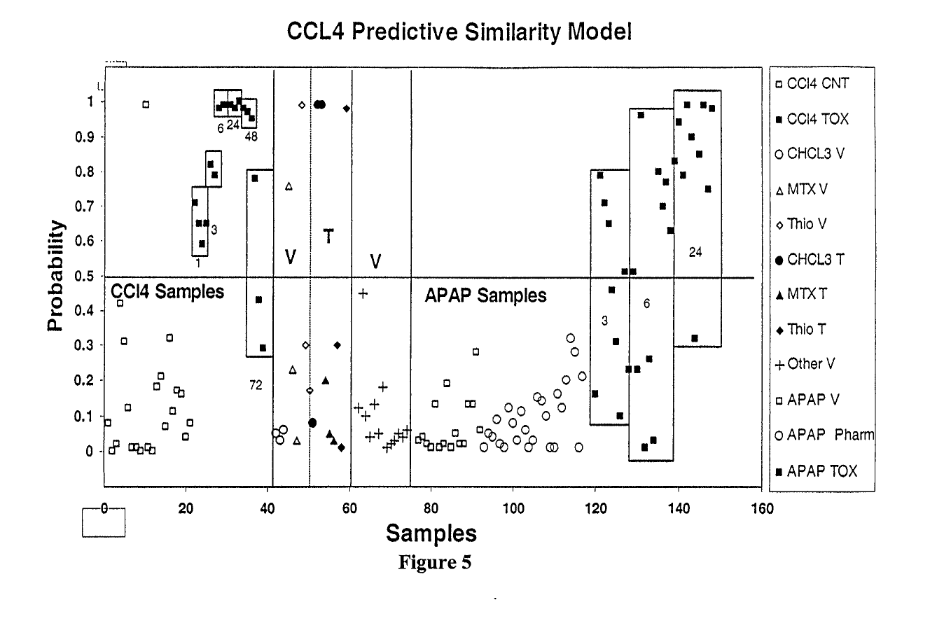

FIG. 5 presents the CCl₄ predictive similarity model.

It will be appreciated that the present invention can be carried out in multiple stages. Specifically, in one preferred embodiment there are four stages of development: selection, quantification, prediction, and validation. In the selection stage, relevant expression patterns that are time stable and dose dependent are determined. In the quantification stage, composite measures that define patterns are produced. In the prediction stage, composite measures to assign probability of similarity of patterns are generated. In the validation stage, statistical measures of model accuracy are provided.

The present invention, a method and system for expression similarity profiling for predictive toxicology, employs a number of different methods for multivariate statistical analysis. In a preferred embodiment, contrast analysis is employed in conjunction with an analysis of variance (ANOVA) for each gene. In this methodology, as input, the average difference for all samples and all genes is generated. Subsequently, an ANOVA analysis is performed.

Analysis of variance (ANOVA) is used to test hypotheses about differences between two or more means. The t-test based on the standard error of the difference between two means can only be used to test differences between two means. When there are more than two means, it is possible to compare each mean with each other mean using t-tests. However, conducting multiple t-tests can lead to severe inflation of the B94286.html Type I error rate. Analysis of variance can be used to test differences among several means for significance without increasing the Type I error rate.

In a preferred embodiment of the present invention using ANOVA analysis, two factors (time, dose) are fitted, using contrast analysis to assign each gene to a pattern. In a particularly preferred embodiment of the present invention, the gene response is fitted to one of a small number of useful patterns. In reality, there are many patterns that could exhibit themselves. This potentially large number of patterns, however, is made up of many simple patterns and only a small number of these patterns are useful in predicting toxicity.

For example if a single does of a drug and a vehicle is administered at three time points. Then, for each time point a gene would demonstrate a basic pattern of either upregulated, downregulated, or not significantly changing. The number of patterns produced would then be three for each time which would mean that 3×3×3=27 patterns can be produced. When we have multiple doses and a larger number of time points, the number of patterns can be extensive. But only a small number of these patterns are useful.

To be useful, a pattern must demonstrate time stability. In that regard, the change in gene expression should go in the same direction for two or more time points and not change direction in adjacent time points relative to the time points where gene expression is changing.

In addition, a useful pattern will preferably demonstrate a dose dependence when multiple doses are used, such as in the APAP model. At the high doses, the pattern must increase or decrease relative to the vehicle and must also increase or decrease from non-toxic doses of that substance in the same direction.

Further, for multiple doses, a general directionality preferably is demonstrated. As the dose increases, the amount of change in gene expression is either increasing or decreasing in the same direction. This can be characterized as a directionality of the pattern in response to an increasing dose.

Thus, the use of contrast analysis permits selection of only those patterns that which are useful with respect to time stability and dose dependence, with a level of confidence in the result based on the appropriate statistical measure (ANOVA).

Upon the conclusion of the analysis, the output provides a list of patterns and a list of genes within each pattern with measures of goodness of fit.

With regard to quantification of the toxicological response, principal component analysis (PCA) is employed. Here for input, genes are selected for patterns that are biologically relevant to the toxicological process. Then, PCA analysis is performed on all samples. The resultant output is 1 to 8 summary scores for each sample.

In the subsequent step, as input, the 1 to 8 summary scores per sample are used as indicators of the toxicity for each sample. In the analysis, a logistical regression analysis maps scores on a 0 to 1 scale of toxicity. The resultant output is a mathematical formula that converts column of summary scores into a single 0 to 1 toxicological score for a sample. With CCl₄, there were 147 patterns generated. 38 patterns with 816 genes were selected. Predictions were based on 4 principal components, with CCl₄ considered toxic at all time points. With APAP, there were 505 patterns generated. 28 patterns with 1024 genes were selected. This was resolved into 8 principal components, with APAP high dose considered toxic at all time points.

| | CCl₄ | APAP |
|---|---|---|
| Percent False Positive | 3/74 (4.1%) | 3/74 |
| (All Samples) | | (4.1%) |
| Percent False Positive | 2/53 (3.8%) | 3/44 |
| (Samples not in Model) | | (6.8%) |

The present invention will allow for the development of models for key compounds; cross-validation of various toxicological models; allow for discrimination of false positive and false negative readings; reduction of toxicological models to a best set of toxic markers; and prediction regarding the toxicity of unknown compounds

The classification of objects into one or more groups based on many measurements has several well established techniques. These include discriminate analysis, logistic regression, multidimensional scaling, clustering, and neural networks. A general discussion of each technique can be found in “Multivariate Analysis, Prentice Hall ISBN 0-13-894858,” which is incorporated herein by reference. All of these methods work by making composite measures from the many measurements taken from each object. With gene expression patterns we have several time and dose points which represent multiple objects that are grouped together. None of these techniques are sufficient alone to represent this order of complexity. Contrast analysis allows us to identify measurements that are partial independent of time because they are time stable yet are affected by toxic doses more then non toxic doses. The PCA combines these many measurements into a series of orthogonal composite measures. Since these composite measures are non correlated by definition the problem of multicolinearity which can decrease the power of logistic regression is eliminated. By combining these techniques in the order described many of the limitations of each individual technique is reduced.

The following is a model developed from gene expression of rat livers using Affymetrix RU35 Rat Chip data. The rats were either treated with a toxic dose, non-toxic dose or vehicle controls. The raw expression data expressed as normalized average differences were then entered into the model described here.

In achieving this analysis, a preferred expression similarity profiling for predictive toxicology algorithm is employed. In this algorithm, let Xij represent gene expression values for the i'th gene and j'th sample (I=1 to I,j=1 to J). Let Yj, Dj, and Tj represent the indicator of toxicity for the j'th sample, the dose for the j'th sample, and the time for the j'th sample, respectively. In the first step, time stable and dose dependent patterns are selected. For gene i, fit a two-factor analysis of variance model. This model can be expressed as

Xij=a|b*Dj|c*Tj|d*Dj*Tj,

for the case of two dose groups (Dj=0 or 1) and two time points (Tj=0 or 1). In this model, the parameters (a, b, c, d) are estimated via a least squares algorithm. Accommodating additional time/dose levels is accomplished by adding additional model parameters for each additional time and/or dose level. For example, the case of four time points (Tj=0 or 1 or 2 or 3) and three dose groups (Dj=0 or 1 or 2) can be expressed as

Xij=a+b1*D1j+b2*D2j+c1*T1j+c2*T2j+c3*T3j+d1*D1j*T1j+d2*D1j*T2j+d3*D1j*T3j+d4*D2j*T1j+d5*D2j*T2j+d6*D2j*T3j,

where T1j=1 if Tj=1, T2j=1 if Tj=2, etc. The parameters (a, b1, b2, c1, c2, c3, d1, d2, d3, d4, d5, d6) are estimated as above.

In the subsequent step, genes are categorized according to the magnitude, sign, and significance level of the estimated parameters. Genes are selected for multivariate statistical analysis of the algorithm if they exhibit dose effects (significant b1, b2, . . . parameters) without time effects (non-significant c1, c2, . . . parameters).

In carrying out the multivariate statistical analysis, the multiple variables are resolved into several components. For the reduced data matrix X′ij (i=genes selected from step 1, j=1 to J), a principal components analysis is performed. The result of this analysis is a series of J principal components, and a score matrix S, where Sij represents the value of the i'th principal component for the j'th sample.

In the next step, a step-up logistic regression procedure is employed, where initially a model with one principal component is fit

Log(Yj/(1−Yj))=a+b1*S1j

The parameters a and b1 are estimated via maximum likelihood estimation. Additional components are added into the model if the model fit would be improved.

This model is used to predict the probability of toxicity for each of the J samples. If the probability for the known toxins is consistently high and the probability for the known non-toxins is consistently low, then the model is accepted. Otherwise, alter the gene selection criteria, and redo the multivariate statistical analysis.

The invention consists of three distinct stages. At each stage, small variations in technique can be used to accomplish the same task. The first stage, selection of time stable and dose dependent patterns by contrast analysis, can be altered by changing the method of measuring variation. We use a method that is based on analysis of variance, where the time component and dose component are assessed simultaneously. One could use a series of t test on individual parts of the pattern to get a collective set of p values that could approximate our method of measuring variation. One could also set an arbitrary fractional cutoff, mean or median of experimental group divided by control group, to approximate the measurement of variation for each part of the pattern that is then use in the next to stages of analysis. The novel feature is to find time. stable and dose dependent patterns with a predicted p value for that pattern.

The second stage, reduction of thousands of variables into one or more composite variables, is accomplished by principal component analysis. Alternative methods exist to produce a composite measure. Partial least squares can be used with control and experimental group being assigned values as dependent variables. Factor analysis has also been used in other settings to reduce many variables into one composite variable.

The third stage, use of composite variables to make one predictive composite measure, is accomplished by entering the principle components, the composite measures from PCA analysis, into a logistic regression. The dependent variable in a logistic regression is the chance of a positive, toxic, or negative, non toxic, outcome that is bounded by the values 1 and 0 respectively. Discriminate analysis could also be used to classify the samples as toxic or non toxic and the discriminate Z scores and distances from the centroids of groups with respect to the Z score variations could be used as alternative method for creating a probability score.

Various preferred embodiments of the invention have been described in fulfillment of the various objects of the invention. It should be recognized that these embodiments are merely illustrative of the principles of the invention. Numerous modifications and adaptations thereof will be readily apparent to those skilled in the art without departing from the spirit and scope of the present invention.

What is claimed is:

1. A method for assessing toxicity and toxicology of a substance, comprising:
exposing a set of at least two genes to the substance;
monitoring the response of each gene in the set of genes to the substance;
analyzing the variance of the response to the substance for each gene using contrast analysis;
constructing a summary score for each gene in the set of genes;
performing a logistic regression analysis upon the summary scores; and
using the results of the logistic regression analysis to provide a predictive model regarding the toxicity and toxicology of the substance.
